(19) United States
(12) Patent Application Publication
(10) Pub. No.: US 2024/0188855 A1
(43) Pub. Date: Jun. 13, 2024

(54) Antenna Assembly for Glucose Monitoring Device

(71) Applicant: KYOCERA AVX Components (San Diego), Inc., San Diego, CA (US)

(72) Inventors: Weichun Eric Lin, San Diego, CA (US); Jin-ho Ahn, San Diego, CA (US)

(21) Appl. No.: 18/517,887

(22) Filed: Nov. 22, 2023

Related U.S. Application Data
(60) Provisional application No. 63/427,619, filed on Nov. 23, 2022.

(51) Int. Cl. A61B 5/145; A61B 5/0507; H01Q 1/27

(52) U.S. Cl. CPC A61B 5/14532; A61B 5/0507; H01Q 1/273

(57) ABSTRACT

Example antenna assemblies for a glucose monitoring device are provided. In one example, the antenna assembly includes an antenna having a planar portion. The antenna is disposed between a circuit board of the glucose monitoring device and a surface of the glucose monitoring device operable to be placed adjacent to a human body during operation of the glucose monitoring device. The planar portion acts as a ground plane for the antenna assembly.

### PRIORITY CLAIM

The present application claims the benefit of priority of U.S. Provisional Application Ser. No. 63/427,619, titled “Antenna Assembly for Glucose Monitoring Device, filed on Nov. 23, 2023, which is incorporated herein by reference.

### FIELD

The present disclosure relates generally to an antenna assembly, and more particularly to an antenna assembly for a glucose monitoring device.

### BACKGROUND

Antennas can be used to facilitate wireless communication between devices. It can be desirable for antennas to operate as with a high antenna radiation efficiency to improve wireless communication between devices. Antennas may need to be incorporated into a variety of different types of devices to provide for wireless communication. With the advance of medical device technology, antennas are being incorporated into a variety of different medical devices, such as glucose monitoring devices.

### SUMMARY

Aspects and advantages of embodiments of the present disclosure will be set forth in part in the following description, or may be learned from the description, or may be learned through practice of the embodiments.

One example embodiment of the present disclosure is directed to an antenna assembly for a glucose monitoring device. The antenna assembly includes an antenna having a planar portion. The antenna is disposed between a circuit board of the glucose monitoring device and a surface of the glucose monitoring device operable to be placed adjacent to a human body during operation of the glucose monitoring device. The planar portion acts as a ground plane for the antenna assembly.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1 depicts an exploded view of an example antenna assembly for a glucose monitoring device according to example embodiments of the present disclosure;

FIG. 2 depicts a stamped metal antenna according to example embodiments of the present disclosure;

FIG. 3 depicts a cross-sectional view of an example glucose monitoring device according to example embodiments of the present disclosure; and

FIG. 4 depicts example antenna radiation efficiency for an antenna assembly according to example embodiments of the present disclosure.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations. As used herein, the use of the term “about” in conjunction with a numerical value is intended to refer to within 10% of the numerical value.

Example aspects of the present disclosure are directed to an antenna assembly for use in, for instance, a glucose monitoring device. Integrating antennas into devices that are worn or placed near a human body can pose challenges due to degradation of RF signals by the human body. According to example embodiments of the present disclosure, the antenna assembly may include a stamped metal antenna. The stamped metal antenna may be operable to be disposed between a circuit board of the glucose monitoring device and a surface of the glucose monitoring device operable to be placed adjacent to a human body during operation of the glucose monitoring device. The stamped metal antenna may have a planar structure that acts as a ground plane for the antenna assembly.

In some embodiments, the circuit board of the glucose monitoring device may have an antenna, such as a trace antenna. The stamped metal antenna may act as a ground plane for the trace antenna. The stamped metal antenna may have planar shape that nearly or generally conforms to a shape of the circuit board. The stamped metal antenna can have an opening configured to accommodate a structure of the glucose monitoring device, such as a sensor operable to be inserted into a human body during operation of the glucose monitoring device. The stamped metal antenna may have a planar portion with a substantially continuous and/or uninterrupted metal. The stamped metal antenna may have one or more tabs to secure the stamped metal antenna to the circuit board. For instance, the stamped metal antenna may have five tabs.

The antenna assembly according to example embodiments of the present disclosure may provide technical effects and benefits. For instance, placing the stamped metal assembly between the circuit board (e.g., with trace antenna) and a surface of a glucose monitor intended to be placed proximate to a human body during operation can cause the stamped metal assembly to act as a ground plane for the antenna assembly. This results in improved antenna radiation efficiency for the antenna assembly at frequencies in, for instance, the 2.4 GHz band.

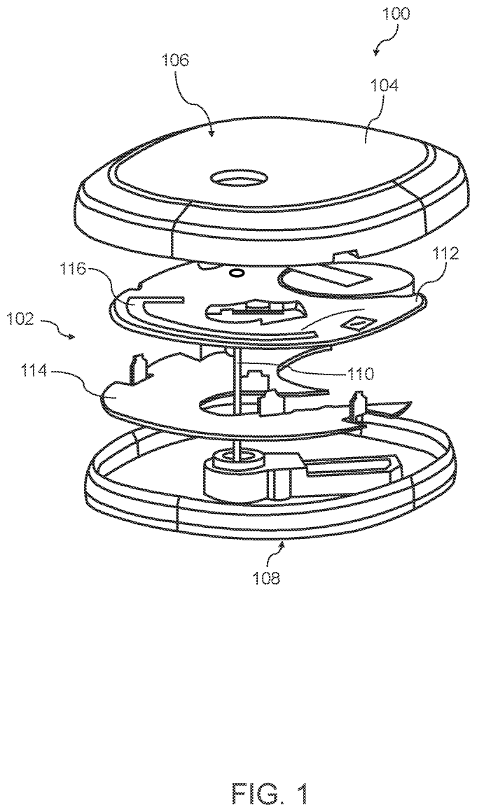

FIG. 1 depicts an exploded view of an example glucose monitoring device 100 including an antenna assembly 102 according to example embodiments of the present disclosure. The glucose monitoring device 100 may include a housing 104, such as a dielectric housing. The housing 104 may have a first surface 106 and a second surface 108 opposite the first surface 106. The second surface 108 may be operable to be placed adjacent to a human body (e.g., directly contacting a human body) during operation of the glucose monitoring device 100. As used herein, the term “directly contact” refers to contact with no intervening structures.

The glucose monitoring device 100 may have a sensor 110. The sensor 110 may extend at least partially from the second surface 108 into or just below the skin of a human body in operation. The sensor 110 may provide for continuous glucose monitoring.

The glucose monitoring device 100 may include a circuit board 112 within the housing 104. The circuit board 112 may include various components/electronics associated with the glucose monitoring device 100.

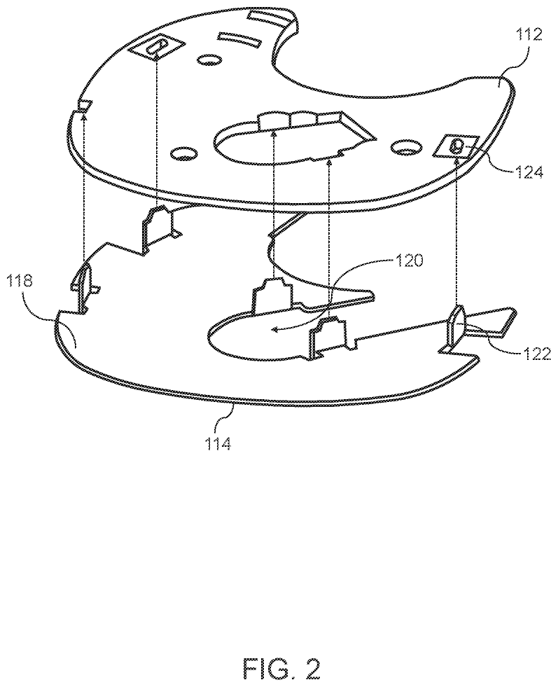

The antenna assembly 102 may include an antenna, such a stamped metal antenna 114. The stamped metal antenna 114 may be disposed between the circuit board 112 and the second surface 108. The stamped metal antenna 114 may include a planar portion (described with reference to FIG. 2) that acts as a ground plane for the antenna assembly 102.

The antenna assembly 102 may include an antenna 116 on the circuit board 112, such as a trace antenna. The stamped metal antenna 114 may include a planar portion (described with reference to FIG. 2) that acts as a ground plane for the antenna 116.

FIG. 2 depicts a close-up exploded view of the circuit board 112 and the stamped metal antenna 114. As illustrated, the stamped metal antenna 114 has a shape that generally conforms to a shape of the circuit board 112. The stamped metal antenna 114 may include a substantially continuous and/or uninterrupted metal planar portion 118. The metal planar portion 118 may be uninterrupted for at least about 90% of the total surface area of the stamped metal antenna 114, such as about 100% of the total surface area of the stamped metal antenna 114. The stamped metal antenna 114 may include an opening to accommodate the sensor 110 (FIG. 1) for the glucose monitoring device 100.

The stamped metal antenna 114 may have one or more tabs 122 to mechanically connect the stamped metal antenna 114 to the circuit board 112. The one or more tabs 122 may be received into openings 124 of the circuit board 112. The example stamped metal antenna 114 of FIG. 2 has five tabs for connection to the circuit board 112.

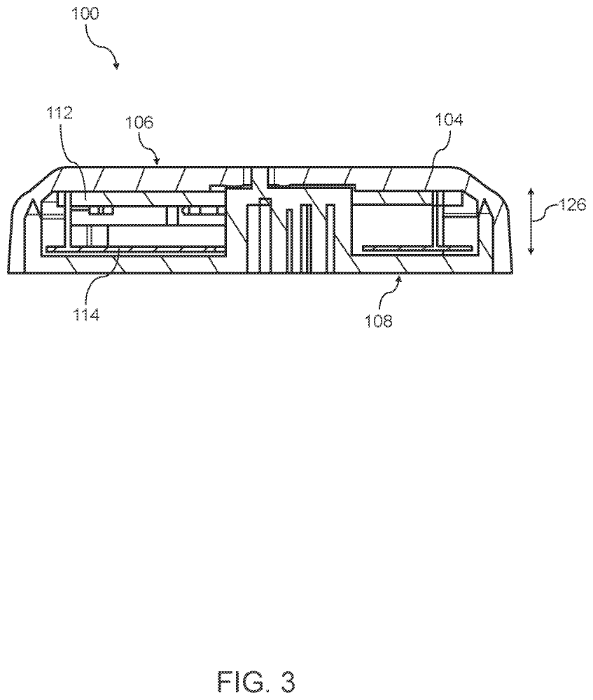

FIG. 3 depicts a cross-sectional view of an example glucose monitoring device 100 according to example embodiments of the present disclosure. As shown in FIG. 3, the stamped metal antenna 114 is separated from the circuit board 112 by a distance 126 when connected to the circuit board 112. In some embodiments, the distance 126 may be less than about 2.9 mm, such as less than about 2.5 mm, such as less than about 2.25 mm, such as in a range of about 1 mm to about 2.5 mm.

As shown in FIG. 3, the stamped metal antenna 114 is disposed in a spaced apart relationship from the circuit board 112 and from an antenna 116 (e.g., trace antenna) disposed on the circuit board 112 in a direction towards the second surface 108. The second surface 108 of the glucose monitoring device 100 is operable to be placed adjacent to a human body during operation of the glucose monitoring device 100.

The antenna assembly 102 (FIG. 1) may be operable to communicate signals in the 2.4 GHz frequency band. In some examples, the antenna assembly 102 may be operable to communicate signals with a remote device (e.g., a smartphone or other device) via a short-range wireless communication protocol (e.g., Bluetooth communication protocol). In some examples, the antenna assembly 102 may be operable to communicate signals with a remote device (e.g., a smartphone or other device) via an 802.11 communication protocol. Other suitable communication protocols may be used without deviating from the scope of the present disclosure.

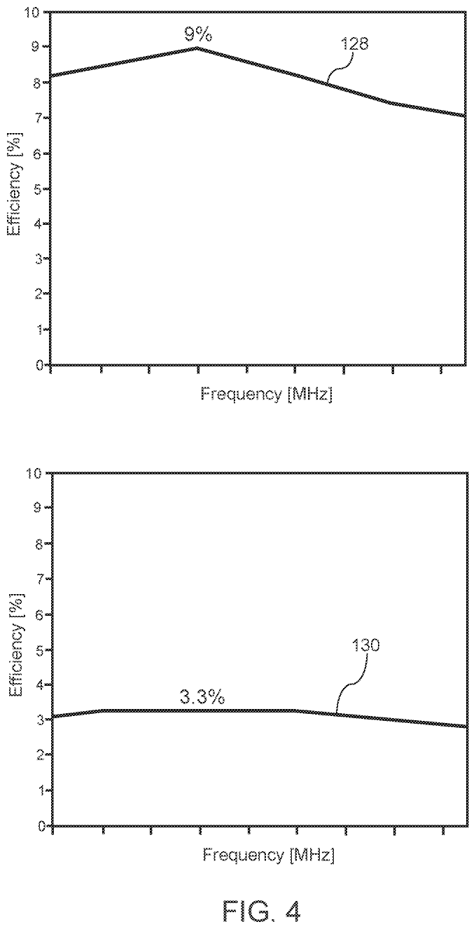

FIG. 4 depicts a plot of example antenna radiation efficiency for an antenna assembly for use with a glucose monitoring device according to example embodiments of the present disclosure. FIG. 4 plots frequencies in the 2.4 GHz frequency band along the horizontal axis. FIG. 4 plots antenna radiation efficiency along the vertical axis. Curve 128 is associated with the antenna assembly 102 wherein the stamped metal antenna 114 is disposed between the circuit board 112 and the second surface 108 of a glucose monitoring device 100 placed adjacent to a human body during operation of the glucose monitoring device 100. Curve 130 is associated with an antenna assembly that is placed between the circuit board 112 and an opposing first surface 106 of a glucose monitoring device that is opposite the surface 108 placed adjacent to the human body.

As illustrated in FIG. 4 by curve 128, the antenna assembly 102 may demonstrate an antenna radiation efficiency of at least about 6%, such as at least about 9% in the 2.4 GHz frequency band. This provides improvement over the antenna represented by curve 130, which only demonstrates an antenna radiation efficiency of about 3.3% in the 2.4 GHz frequency band.

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. An antenna assembly for use in a glucose monitoring device, comprising:
an antenna comprising a planar portion;
wherein the antenna is disposed between a circuit board of the glucose monitoring device and a surface of the glucose monitoring device operable to be placed adjacent to a human body during operation of the glucose monitoring device; and
wherein the planar portion is operable to act as a ground plane for the antenna assembly.

2. The antenna assembly of claim 1, wherein the antenna is a stamped metal antenna.

3. The antenna assembly of claim 1, further comprising a trace antenna on the circuit board.

4. The antenna assembly of claim 3, wherein the planar portion acts as a ground plane for the trace antenna.

5. The antenna assembly of claim 1, wherein the antenna comprises a plurality of tabs to connect the antenna to the circuit board.

6. The antenna assembly of claim 1, wherein the planar portion has an opening to accommodate at least a portion of a glucose sensor at least partially extending from the glucose monitoring device.

7. The antenna assembly of claim 1, wherein the planar portion is separated from the circuit board by a distance when connected to the circuit board.

8. The antenna assembly of claim 7, wherein the distance is less than about 2.9 mm.

9. The antenna assembly of claim 1, wherein the antenna assembly is operable to communicate signals over a 2.4 GHz frequency band.

10. The antenna assembly of claim 9, wherein the 2.4 GHz frequency band is associated with a short-range wireless communication protocol to a remote device.

11. The antenna assembly of claim 9, wherein the 2.4 GHz frequency band is associated with an 802.11 communication protocol to a remote device.

12. The antenna assembly of claim 1, wherein the antenna assembly demonstrates an antenna radiation efficiency of at least about 6% in a 2.4 GHz frequency band.

13. The antenna assembly of claim 1, wherein the antenna assembly demonstrates an antenna radiation efficiency of at least about 9% in a 2.4 GHz frequency band.

14. A glucose monitoring device, comprising:
a housing having a first surface and a second surface opposite the first surface;
a glucose sensor at least partially extending from the second surface;
a circuit board disposed in the housing, the circuit board comprising a first antenna; and
a stamped metal antenna disposed between the circuit board and the second surface.

15. The glucose monitoring device of claim 14, wherein the first antenna is a trace antenna.

16. The glucose monitoring device of claim 14, wherein the stamped metal antenna is operable to act as a ground plane for the first antenna.

17. The glucose monitoring device of claim 14, wherein the stamped metal antenna has a continuous planar portion over at least about 90% of a total surface area of the stamped metal assembly.

18. The glucose monitoring device of claim 17, wherein the continuous planar portion has an opening to accommodate the glucose sensor extending at least partially from the second surface.

19. An antenna assembly for a glucose monitoring device, comprising:
a first antenna disposed on a circuit board;
a stamped metal antenna disposed in spaced apart relationship from the first antenna towards a surface of the glucose monitoring device placed adjacent to a human body during operation of the glucose monitoring device; and
wherein the antenna assembly has an antenna radiation efficiency of at least about 6% in the 2.4 GHz frequency band.

20. The antenna assembly of claim 19, wherein the first antenna is a trace antenna.
